(19) United States
(12) Patent Application Publication
(10) Pub. No.: US 2015/0285778 A1
(43) Pub. Date: Oct. 8, 2015

(54) TEST WAND FOR LIQUIDS

(71) Applicant: HACH COMPANY, Loveland, CO (US)

(72) Inventors: Matthew P. Hertel, Granger, IN (US); David A.N. Morris, Goshen, IN (US)

(21) Appl. No.: 14/441,781

(22) PCT Filed: Nov. 12, 2013
(86) PCT No.: PCT/US13/69700

§ 371 Date: May 8, 2015

Related U.S. Application Data
(60) Provisional application No. 61/724,822, filed on Nov. 9, 2012.

(51) Int. Cl. G01N 33/18; G01N 21/78; G01N 21/84; G01N 21/80

(52) U.S. Cl. CPC G01N 33/18; G01N 21/80; G01N 21/78; G01N 21/8483; G01N 2021/8488; G01N 2021/7793

(57) ABSTRACT

A species test wand for liquids, wherein a user is informed whether the concentration of a species is high, at an acceptable level, or low by a comparison of the color of the wand after exposure to the liquid with nearby labeled colored boxes integral with the wand, is described. Tests include liquid evaluation for chlorine, pH and total alkalinity, as examples.

### CROSS-REFERENCE TO RELATED APPLICATIONS

The present application claims the benefit of U.S. Provisional Patent Application No. 61/724,822 for “Indicator And Test Wand For Liquids,” which was filed on Nov. 9, 2012, the entire content of which application is hereby specifically incorporated by reference herein for all that it discloses and teaches.

### FIELD OF THE INVENTION

Embodiments of the present invention relate generally to test strips for determination of the concentration of specific ions or other species in liquid samples and, more particularly, to a test wand having comparison color boxes integrated onto the wand for each test strip for indicating whether a particular species is present in amounts which are too high, too low, or acceptable.

### BACKGROUND OF THE INVENTION

Generally, strips of filter paper impregnated with indicator compositions sensitive for specific species, are used to test liquids for these species. After contacting the strip with the liquid, the strip is compared with a color chart having numerical labels relating the color to species concentration, or to pH in the case of hydronium ions. Such charts may be located on the strip packaging or on a separate color sheet. Once the concentration of the species is established, the tester evaluates the numbers to determine whether remedial action must be taken and, if so, what action.

### SUMMARY OF THE INVENTION

Embodiments of the present invention overcome the disadvantages and limitations of the prior art by providing a test for at least one species in a liquid, wherein the user is directly informed whether the concentration of the species is high, at an acceptable level, or low.

Another object of embodiments of the invention is to provide a test for at least one species in a pool or spa, wherein the user is directly informed whether the concentration of the species is high, at an acceptable level, or low.

Still another object of embodiments of the invention is to provide a test for at least one species in a pool or spa, wherein the user is directly informed whether the concentration of the species is high, at an acceptable level, or low, without color matching.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

To achieve the foregoing and other objects and in accordance with the purposes of the present invention, as embodied and broadly described herein, the test wand for determination of species concentration in a liquid, hereof includes: a substrate; at least one band having a species-specific test composition thereon effective for producing a particular color in the presence of a specific species concentration range and mounted on said substrate; and at least one labeled color comparison box corresponding to each of the at least one test bands integrated onto the substrate and disposed in the vicinity of the at least one test band such that the color of the at least one test band is readily compared with the color of the at least one comparison box; whereby, color is produced on the at least one test band when the wand is immersed in the liquid.

Benefits and advantages of embodiments of the present invention include, but are not limited to, providing a species test wand for evaluating the concentration of chosen species in a liquid and indicating the action needed to be taken by the user without the user having to compare the color of a band on the wand with a separate color chart after contact with the liquid, and without having to evaluate numbers typically associated with such charts as labels.

### BRIEF DESCRIPTION OF THE DRAWINGS

The accompanying drawings, which are incorporated in and form a part of the specification, illustrate an embodiment of the present invention and, together with the description, serve to explain the principles of the invention. In the drawings:

FIG. 1A is schematic representation of a test wand illustrating bands of compositions effective for displaying colors in response to concentration ranges of species under investigation, such as total alkalinity, TA, pH, and chlorine, Cl, concentration, as examples, each band having a corresponding color boxes indicating whether the concentration of that species is high, at an acceptable level, or low, while FIG. 1B illustrates the wand being dipped into a liquid containing at least one species to be evaluated.

### DETAILED DESCRIPTION OF THE INVENTION

Briefly, embodiments of the present invention include a species test wand for liquids, wherein the user may be informed whether the concentration of a particular species is high, at an acceptable level, or low by comparing the color of test strips disposed on the surface of the wand after exposure to the liquid with nearby associated and labeled colored boxes on the wand. The test wands hereof find use for testing water in pools and spas, as examples.

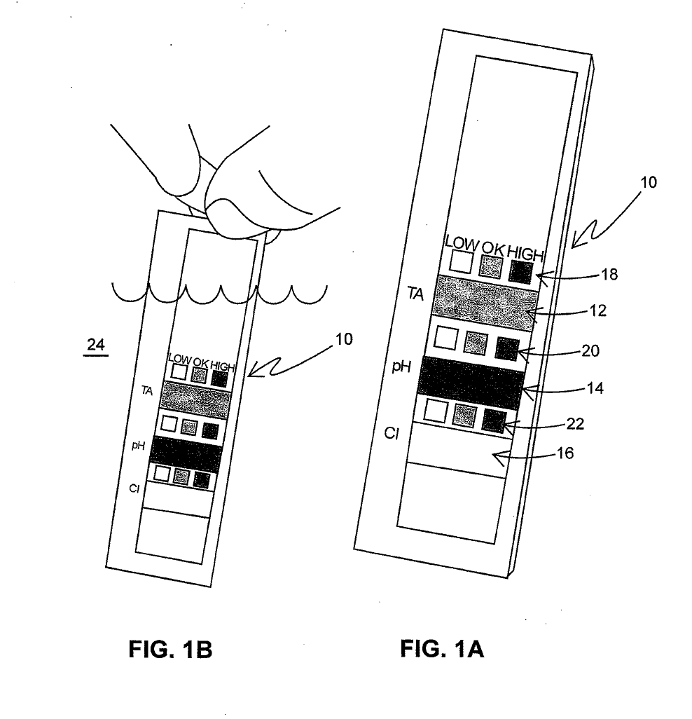

Reference will now be made in detail to the present embodiments of the invention, examples of which are illustrated in the accompanying drawings. In the FIGURES, similar or identical structure will be identified using identical reference characters. It will be understood that the FIGURES are for the purpose of describing particular embodiments of the invention and are not intended to limit the invention thereto. Turning first to FIG. 1A, shown is a schematic representation of test wand, 10, shown as being elongated, illustrating at least one band or test strip, and shown as bands 12, 14, and, 16, of compositions effective for displaying colors in response to concentration ranges of at least one species under investigation, such as total alkalinity, TA, pH (related to the concentration of hydronium ions in solution), and chlorine, Cl, concentrations, respectively, each band having a series of corresponding labeled colored boxes, 18, 20, and 22, respectively, located nearby to or adjacent to their associated band on wand 10 for indicating whether the concentration of material is high, at an acceptable level, or low, while FIG. 1B illustrates wand 10 being dipped into liquid, 24, containing at least one species to be tested. As an example, the term “corresponding” means that the series of boxes for a pH measurement have colors relating to chosen pH values or ranges of values indicative of these labels. Since the user is informed as to the condition of the at least one species in the liquid sample, it is immediately clear what action need be taken by the user. For example, if the water sample is taken from a swimming pool, and the wand result is that the pH is high, the user is informed by the test wand after the test that agents have to be added to the pool to lower the pH value. Tests for different species may be simultaneously conducted by dipping all of the bands into the liquid at the same time.

The reactive bands include cellulose and non-cellulose fibrous materials impregnated with indicators sensitive to specific species in the liquids being tested, and mounted on a substrate using an adhesive. Impregnated filter paper strips mounted on a polystyrene wand using double-sided adhesive paper is an example of such bands.

Colored comparison boxes are disposed in the vicinity of or adjacent to the reactive test areas, thereby providing a convenient means for color comparison and determination of the test levels for each chemistry, as may be observed from FIG. 1A, where the colors are illustrated using gray tones. The boxes may be integrated onto the wand by printing using insoluble inks, or the printed boxes may be coated with a transparent, water impervious coating to protect the boxes from the liquids undergoing testing. If the wand is made of hydrophobic material, the ink may be hydrophobic as well and not subject to degradation by the liquid sample for the short wand submersion times.

As an example, labels may include: “LOW” for indicating a concentration lower than recommended, “OK” for indicating a suitable concentration, and “HIGH” for indicating a concentration that is above the recommended range. Shown also in FIG. 1A are identification letters for the test bands, Total Alkalinity (“TA”), “pH” and free chlorine “Cl”. FIG. 1B illustrates the subject wand being placed in the liquid for which these species are simultaneously being measured. As an example, in FIG. 1B, the “TA” band is seen to correspond to the “OK” box, “pH” with the “HIGH” box, and “Cl” with the “LOW” box.

Clearly, multiple bands having identical reactive chemistry, pH, as an example, may be placed on a wand for sequential tests of a liquid using a single wand, by dipping only one unused test band at a time into the liquid.

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A test wand for determination of species concentration in a liquid, comprising:
a substrate;
at least one band having a species-specific test composition thereon effective for exhibiting a particular color in the presence of a specific species concentration range and mounted on said substrate; and
at least one labeled color comparison box corresponding to each of said at least one test band integrated onto said substrate and disposed in the vicinity of said at least one test band such that the color of said at least one test band is readily compared with the color of the at least one comparison box;
whereby, color is produced on said at least one test band when said wand is immersed in said liquid.

2. The test wand of claim 1, wherein said at least one test band is mounted using an adhesive.

3. The test wand of claim 1, wherein said substrate comprises polystyrene.

4. The test wand of claim 1, wherein said tests are chosen from chlorine, pH and total alkalinity.

5. The test wand of claim 1, wherein said at least one color comparison box comprises boxes labeled as high, acceptable and low for said species.

6. The test wand of claim 1, wherein said wand is elongated.

7. The test wand of claim 6, wherein said at least one test band comprises multiple bands having the same composition disposed along the long dimension of said substrate, such that several liquid tests are conducted with said wand.

8. The test wand of claim 1, wherein said liquid comprises liquid from a pool or a spa.
